(19) United States
(12) Patent Application Publication
(10) Pub. No.: US 2005/0171405 A1
(43) Pub. Date: Aug. 4, 2005

(54) Surgical retractor with intermediate support members

(76) Inventors: Randall P. Rowland, Eden Prairie, MN (US); Hans A. Mische, St. Cloud, MN (US)

Correspondence Address: Kagan Binder, PLLC;Maple Island Building, Suite 200, 221 Main Street North, Stillwater, MN, 55082, US

(21) Appl. No.: 11/031,054

(22) Filed: Jan. 7, 2005

Related U.S. Application Data
(60) Provisional application No. 60/535,012, filed on Jan. 8, 2004.
(60) Provisional application No. 60/535,011, filed on Jan. 8, 2004.

(51) Int. Cl. A61B 1/32

(52) U.S. Cl. 600/233

(57) ABSTRACT

A surgical retractor system is provided comprising an annular frame having an inner edge and an outer edge having a plurality of spaced notches, wherein the inner edge defines an inner area of the frame, and at least one support member having a first end, a second end, and first and second edges extending between the first and second ends, wherein the first and second ends are attached to the annular frame so that at least a portion of the support member between the first and second ends extends over at least a portion of the inner area of the frame, and wherein at least one of the first and second edges comprises at least one notch.

### REFERENCE TO RELATED APPLICATIONS

This application claims the benefit of U.S. Provisional applications having Ser. No. 60/535,012, filed Jan. 8, 2004, entitled “SUPPORT STRAP IMPROVEMENTS”; and Ser. No. 60/535,011, filed Jan. 8, 2004, entitled “SURGICAL RETRACTORS”, which applications are incorporated herein by reference in their entireties.

### TECHNICAL FIELD

The present invention relates to surgical instruments and more specifically to surgical retractors for use in various surgical procedures that require access to internal body organs or tissues by a surgeon.

### BACKGROUND OF THE INVENTION

During many surgical procedures, an incision is made through the skin and into the tissue of the patient in order to provide access to internal organs. The surgical incision is then separated and retracted in some way to maintain the opening in an open or exposed condition to thereby provide access to the operating site by a surgeon. In some cases, as the surgeon cuts into the tissue, the operating room staff will hold the tissue away from the operating field using retractors. One way this is accomplished is with the use of one-piece metallic retractors that typically retract the wound in a non-yielding manner that can result in tearing and bruising of the tissue. Another way retraction is accomplished is through the use of “stay” sutures that are placed through the tissue. These sutures are typically controlled by clamping a device such as a hemostat to the end of each of the sutures so that the hemostats or other devices can be pulled in the desired directions to widen the incision via the movement of the sutures. The sutures then can also maintain the tissue in an open condition. The site is then more readily accessible by the surgeon for reaching the affected organs, tissues, muscles, and the like, for performing the necessary surgery and/or for the implantation of various devices into the body. However, the use of these sutures and attached devices can clutter the surgical field for the surgeon and can be difficult to maintain in their desired positions due to the variations in the skills of the operating staff, especially in cases where the surgery takes an extended period of time.

Thus, various types of retractors that require less constant interaction by the operating staff are available for different types of surgeries, where the devices are designed to accommodate the particular area of the body on which surgery will be performed. These retractors are typically designed to not obstruct either visual or physical access by the surgeon. For example, retractors used in spinal surgery require a retractor that is strong enough to overcome a relatively large muscle mass that needs to be dissected away from the field of exposure, such as a retractor that includes large blades or paddles that move the muscles and tissues away from the spine to provide the necessary access by the surgeon.

In other cases, a retractor frame may be designed to specifically conform to the portion of the body where the surgery will be performed, which may be used with multiple retractor devices or stays. One particular type of frame that may be used for penile-scrotal surgeries that require dissection and exposure of the corpora includes a retractor frame that is particularly designed for the genital area of the body. The retractor frame can be placed against the skin of the patient around the surgical site either before or after the incision is made by the surgeon. Elongated retractor stays, which are typically made of an elastic material, can then be positioned so that one end of the stay engages with the tissue at the incision and the other end is attachable to the retractor frame. These retractor stays may be repositioned throughout the surgery, as desired, to provide adequate access to the surgical site for the surgeon. In one particular retractor frame design, the frame is provided with a plurality of notches spaced about the periphery of the frame, while the stays include a tissue-holding device (e.g., a hook portion) at one end of an elastic member. The surgeon can position the tissue-holding device within the incision, then use the elastic member to adjust the traction applied to the tissue by the placement of the elastic member within the notches. Retractor frame designs known in the art include those having rigid, one-piece constructions that are contoured generally to fit a particular area of the human body, and those frames that include portions that are moveable relative to one another (e.g., two portions that pivot about two pivot points). Other retractor frames are more capable of being adjusted or reconfigured to match the contours of the surgical site, such as with constructions having two or more pieces with malleable portions connecting them to each other.

Regardless of the particular adjustability of the frames, they are typically provided in an annular shape, with the center area being open to provide the maximum amount of access to the surgical area for the surgeon. There is, however, a need to provide a retractor frame that includes additional support in the surgical site during surgery and that accommodates the anatomy of the patient.

### SUMMARY OF THE INVENTION

In one aspect of this invention, a surgical retractor system is provided comprising an annular frame having an inner edge and an outer edge having a plurality of spaced notches, wherein the inner edge defines an inner area of the frame, and at least one support member having a first end, a second end, and first and second edges extending between the first and second ends, wherein the first and second ends are attached to the annular frame so that at least a portion of the support member between the first and second ends extends over at least a portion of the inner area of the frame, and wherein at least one of the first and second edges comprises at least one notch. At least one notch of the support member may be engaged with at least one notch in the outer edge of the annular frame. At least one notch of the support member may be in the portion of the support member that extends over at least a portion of the inner area of the frame. In another alternative, the first end of the support member may be permanently attached to the annular frame and the second end of the support member may be repositionable relative to the first end of the support member. At least one notch of the support member may increase in width in response to stretching the support member, and the support member may comprise notches on both the first and second edges. Also, the support member may comprise a curved portion.

In another aspect of the invention, a method of positioning a surgical retractor system for a surgical procedure is provided, comprising the steps of providing an annular frame having an inner edge and an outer edge having a plurality of spaced notches, wherein the inner edge defines an inner area of the frame, attaching a support member having a first end, a second end and first and second edges extending between the first and second ends to the annular frame so that at least a portion of the support member between the first and second ends extends over at least a portion of the inner area of the frame, wherein at least one of the first and second edges comprises a notch; and positioning the annular frame with attached support member over a surgical area of a patient.

### BRIEF DESCRIPTION OF THE DRAWINGS

The present invention will be further explained with reference to the appended Figures, wherein like structure is referred to by like numerals throughout the several views, and wherein:

FIG. 1 is a top plan view of a surgical frame including a support member or platform, in accordance with the present invention;

FIG. 2 is a front schematic view of a platform or support member connected to a surgical frame with a gate-type connection;

FIG. 3 is a front schematic view of another embodiment of a platform or support member connected to a surgical frame with a snap-fit type of connection;

FIG. 4 is a top plan view of a surgical frame including another embodiment of a support member or platform;

FIG. 5 is a top plan view of an embodiment of a support strap for use with a surgical frame in accordance with the present invention;

FIG. 6 is a top plan view of another embodiment of a support strap for use with a surgical frame;

FIG. 7 is a top plan view of another embodiment of a support strap for use with a surgical frame; and

FIG. 8 is a side view of another embodiment of a support strap for use with a surgical frame.

### DETAILED DESCRIPTION OF THE PREFERRED EMBODIMENTS

Referring now to the Figures, wherein the components are labeled with like numerals throughout the several Figures, and initially to FIG. 1, one preferred configuration of a surgical frame 10 is shown. Surgical frame 10 generally includes a first portion 12 that is generally a truncated portion of a circular or arcuate shape, and a second portion 14 that is somewhat smaller in diameter than first portion 12, but is also generally a truncated portion of a circular or arcuate shape. The two portions 12, 14 are positioned relative to each other to make a generally annular frame 10 having an inner area 16 in which the surgical procedure will be performed, as explained in further detail below. The peripheral shape of the frame 10 is generally a “figure 8”, as shown; however, it is understood that the peripheral shape of the two portions 12, 14 can vary considerably in shape and size from that shown to form a different outer peripheral shape of the frame 10. For example, the two portions 12, 14 may be the same size as each other, one or both portions 12, 14 may be more oblong or oval in shape, one or both portions 12, 14 may be more square or rectangular in shape, one or both portions may be more V-shaped, or the like. In any case, the size and shape of the first and second portions 12, 14 should be chosen to accommodate the general contours of the portion of the body on which it will be positioned.

The surgical frame 10 has an upper surface 18 and an opposite lower surface (not shown). The upper surface 18 is substantially planar and extends from an inner edge 20 to an outer edge 22. The upper surface 18 may be substantially flat, but preferably angles somewhat upwardly from the inner edge 20 to the relatively higher outer edge 22. Alternatively, the upper surface 18 may be curved generally upwardly from the inner edge 20 to the outer edge 22, or the surface 18 may extend at one angle or curve from the inner edge 20 to an inner ridge 24, then extend at another angle or curve from the inner ridge 24 to the outer edge 22. The surface 18 may alternatively have different contours or angles provided to conform generally to the contours of the portion of the body on which the frame 10 will be used.

The outer edge 22 includes a plurality of notches 26 that extend through the frame 10 and are spaced around its periphery. As shown, the notches 26 are evenly spaced from each other; however, the notches 26 may instead be spaced at different intervals from each other around the periphery of the surgical frame 10. The notches are designed to accommodate the efficient organization of elastic stay hooks, when such hooks are desired for a particular surgical procedure. Examples of stay hooks that can be used with the retractor frames of the invention include the elastic stay hooks commercially available from Lone Star Medical Products, Inc. of Stafford, Tex., which generally include an elongated elastic band having a hook or multiple hooks at one end for engagement with the tissue surrounding the incision. The hooks can be small or large, may include single or double hooks, may be sharp or blunt, may include solid blades, and/or may include blunt rakes with two or more fingers, as desired for the particular surgery.

The second frame portion 14 preferably further includes extension portions 28 extending from each end of the truncated circular frame portion 14, which are shown in the figure as being generally horizontal and parallel to the other extension portion 28 on the opposite side of the frame portion 14. These extension portions 28 preferably include a relatively flat piece that extends in a generally perpendicular direction from the plane of the frame portion 14. Similarly, the first frame portion 12 includes extension portions 30 extending from each end of the truncated circular frame portion 12, which are shown in the figure as being generally horizontal and parallel to the other extension portion 30 on the opposite side of the frame portion 12. These extension portions 30 also preferably include a relatively flat piece that extends in a generally perpendicular direction from the plane of the frame portion 12. As shown, the extension portions 28 are located closer to the inner area 16 of the frame than the extension portions 30, and the planar surfaces of extension portions 28 are preferably designed for contact with the planar surfaces of the extension portions 30. The extension portions 28, 30 may be molded into the second or first frame portions 14, 12, respectively, in a unitary-piece construction, or the extension portions 28, 30 may instead be separate pieces that are attached to frame portions 14, 12, respectively, such as by gluing, welding, or the like.

Each of the extension portions 28, 30 preferably include a connection hole through their thicknesses (not shown) that may optionally be threaded, or may be a simple through-hole. As shown, the extension portions 28 of the frame overlap the extension portions 30 so that their connection holes are aligned for accepting a connection member 32 on each side of the frame. In one embodiment of the invention, the connection members 32 are threaded to mate with threads inside the connection holes so that the connection members 32 can be loosened to allow relative movement between the first frame portion 12 and the second frame portion 14, then can be tightened once the frame portions 12, 14 are in their desired positions relative to each other by rotating the wing portion of the connection members 32 to fix the extensions 28, 30 in a final position and lock the frame in position. A variety of other configurations for locking the first and second frame portions 12, 14 relative to each other are contemplated, such as using a malleable connector between the pieces, using connection members that are threaded only at one end to accept a nut that can be tightened to lock the frame portions 12, 14 relative to each other, and the like. Further, the extension portions 28, 30 may extend at different angles than perpendicular to their respective frame portions 14, 12, but preferably are designed to allow the two portions 14, 12 to move relative to each other, then be locked into place when a desired orientation is achieved.

While the above discussion of the frames of the present invention include two frame portions that pivot relative to each other, the frames may instead be a single rigid piece that incorporates a platform or support member within its inner area, or the frame may include more than two pieces that pivot or rotate relative to each other. For example, the first portion 12 of frame 10 of FIG. 1 may itself comprise two portions that are hinged relative to each other along the periphery of the portion 12 and may or may not also include the various support structures described above relative to the structures positioned at the pivot area between the first and second portions 12, 14. Further, the entire peripheral structure of the frames described above may be malleable or otherwise reconfigurable, or selected portions of the peripheral structure of the frames of the present invention may be malleable or otherwise configurable while other portions are rigid or semi-rigid, such as in the area where pivoting occurs, for example.

The surgical or retractor frames may be reusable and made of a material that can be sterilized at the point of use, such as aluminum, stainless steel, titanium, or other medical-grade metals. Alternatively, the frames may instead be disposable and therefore made of a material that is not designed to be sterilizable at the surgical site, such as a plastic material that is sufficiently strong to support the use of any types of stays that are connected thereto without causing bending or buckling of the frame. One example of such a material from which a disposable frame may be made is a plastic or resin material, and one preferred example of which is a polyphenylene ether/polystyrene blend (PPE/PS) commercially available under the trade name “Noryl” and available from GE Plastics of Pittsfield, Mass. These disposable surgical frames can be pre-sterilized and packaged singly in pouches for delivery to the sterile surgical field, and typically weigh less than similarly shaped and sized reusable frames, but should still be made of a material that provides adequate structural rigidity for the surgeon.

The surgical frame 10 further preferably includes a platform or support member 34 that extends generally from the extension portions 28, 30 on one side of the frame 10 to the extension portions 28, 30 on the opposite side of the frame 10. In this configuration, the support member 34 extends across the inner area 16, essentially dividing the inner area 16 into two portions. In the case of penile-scrotal surgical procedures, this support member 34 can be positioned generally at the base of the penis at the pubic region, thereby improving the support and positioning of the penis during surgery. That is, when the penis is directed toward the patient's abdomen, the platform or support member 34 will be under the penis, which will help to stabilize the penis and provide a firm platform to aid the surgeon in the surgical procedure.

The support member 34 is shown in FIG. 1 as an elongated bar or tube that connects at both ends to the extension portions 28, 30 of the frame portions 12, 14, but is referred to herein as attaching to the frame portions 12, 14 because the exact location for the attachment can vary from that shown. The support member 34 may have a slot or opening at each end that slides, snaps, or otherwise attaches to the frame portions 12, 14. The frame portions 12, 14, may include additional and/or separate pieces or portions for attachment of the support member 34, but the support member 34 is preferably designed for use with frames 10 that are configured generally as shown (i.e., with extension portions 28, 30). Thus, a wide variety of configurations are possible for both the support member 34 and the portion of the frame 10 to which it is attached.

FIG. 2 illustrates one configuration of an attachment of a support member 34a to an extension portion 28a of a frame, similar to extension portions 28 on opposite sides of frame 10. As shown, the support member 34a is configured as a “gate” where it contacts attachment points 36 on both of its ends adjacent to the extension portions 28a. In this embodiment, the support member 34a can be rotated or pivoted about one or both of the attachment points 36 into either a closed or open position, depending on the surgical requirements. For example, it may be desirable to have the support member 34a “closed” or rotated into place within the inner area of the frame during the surgical process, but have the support member “open” or rotated out of the inner area of the frame to provide a more open area for the suturing process. Thus, the support member 34a can preferably rotate or pivot about either of the attachment points 36, although it is understood that the support member 34a may rotate about only one of the attachment points 36, with the other end of the support member 34a functioning as a “free end” that moves into and out of place, as desired. In the case where the support member 34a rotates about only one attachment point 36, the opposite end of the support member 34a can preferably snap or otherwise lock into one of the extension portions 28a of the frame, or other area of the frame with which it comes in contact.

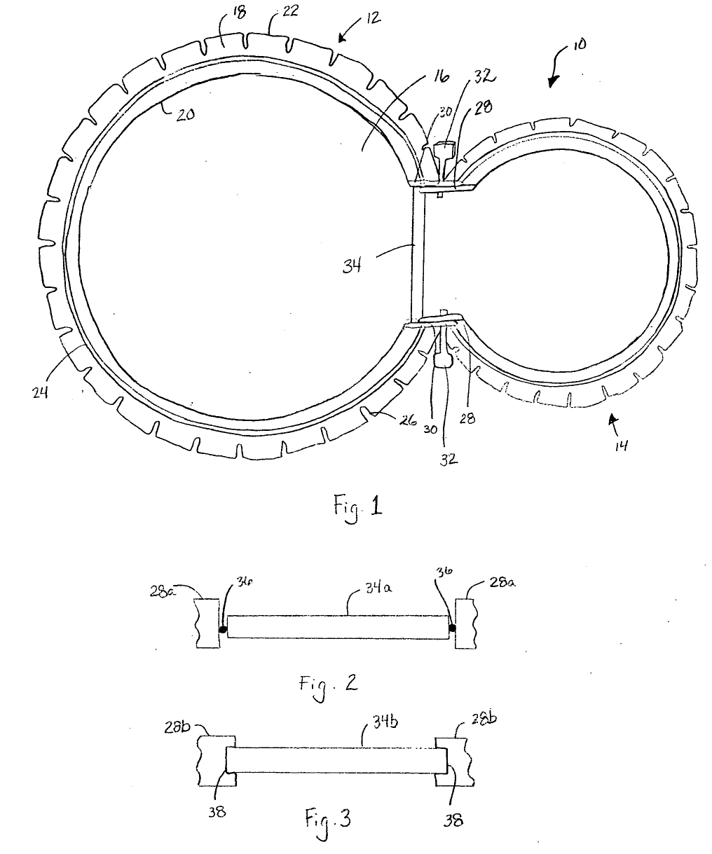

FIG. 3 illustrates another configuration of an attachment of a support member 34b to an extension portion 28b of a frame, similar to extension portions 28 on opposite sides of frame 10. In this case, the support member 34b is a completely separable component from the frame to which it will attach. As shown, the extension portions 28b each include a notch or recessed area 38 that is closely sized to accept an end of the support member 34b. In this case, the support member 34b can be inserted into the notches 38 when the surgeon desires to use the support member 34b during the surgical procedure, then the support member 34b can be removed if desired to provide more open access to the inner area of the frame. For example, it may be desirable to have the support member 34b positioned within the notches 38 to be within the inner area of the frame during the surgical process, but have the support member removed from the inner area of the frame to provide a more open area for the suturing process. The notches 38 in the extension portions 28b are preferably designed to securely hold the support member 34b in place, therefore, the notches should be sufficiently large that external pressure on the support member 34b will not unintentionally dislodge the support member 34b from the notches 38.

The support member 34b may be inserted into the notches 38 of the extension portions 28b in a number of ways, depending on the particular design of the components and the materials from which they are made. One installation technique includes inserting one end of the support member 34b into one of the notches 38 from the top of the device (e.g., from the upper surface 18 toward the bottom surface of frame 10), then pressing downward on the opposite end of the support member 34b to slightly deflect one or both of the extension portions 28b away from the opposite extension portion 28b. This opposite or free end of support member 34b will continue to be pushed downward until it is in line with the notch 38 in the adjacent extension portion 28b. At this point, the support member 34b will slip or snap into the notch 38, thereby allowing the extension portions 28b to return to their original positions relative to each other and retain the support member 34b in place. Another possible installation technique is similar to the previously described technique, but in this case both ends of the support member 34b are simultaneously pressed downward to deflect the extension portions 28b away from each other. The support member 34b will continue to be pressed downward until both ends of the support member 34b snap or slip into the notches 38 of their respective adjacent extension portions 28b to retain the support member 34b in place between the extension portions 28b. Yet another exemplary installation technique involves slipping the support member 34b into the notches 38 from the side of the extension portions 28b (e.g., from the side of the first frame portion 12 toward the second frame portion 14 in FIG. 1). This technique does not require any deflection of the extension portions 28b.

In cases where the support member is provided as a separate component from the surgical frame, it may be advantageous to provide a frame system including several support members from which a surgeon can choose for a particular surgery and patient. This type of system would be particularly advantageous for reusable frames so that the surgeon could use custom designed frames with or without support members for multiple unique surgeries. However, systems including a choice of several support members can also be useful for disposable frame systems so that the surgeon can choose the type of support member that would best suit a particular surgery at the time of surgery for a frame that is customized to the patient.

Referring again to FIG. 1, the support member 34 may be a permanent part of the structure of the frame 10 that is not detachable or partially detachable as described relative to FIGS. 2 and 3. In the permanent installation, it is preferable that the support member 34 be connected in such a location that it does not interfere with any rotation of frame portions relative to each other. The support member 34 may be provided as a separate piece that is permanently attached to the desired portions of the frame 10 by welding, gluing, or other attachment method that is appropriate for the materials from which the components are made. Alternatively, the support member 34 may be permanently connected to the frame with some sort of mechanical connection such as a snap connector that is not removable once the support member 34 is in place. It is further contemplated that the support member 34 be integrally molded with a portion of the frame 10 so that the support member 34 is a part of a unitary frame construction.

The support member 34 may have a generally circular cross-section, or may include any of a wide variety of other shapes that are preferably relatively easy to manufacture. Other cross-sectional shapes for the support member 34 include square, rectangular, oval, triangular, hexagonal, and the like. In one particular example of a rectangular or oval cross section, at least a portion of the support member 34 may be relatively flat, such as in the form of a strap with an oval or rectangular cross-section. Other cross-sections are also possible for this strap-like configuration. The cross-section can also be relatively complex in shape, particularly when desired for particular surgical processes.

The support member 34 may be either solid or hollow along all of its length, or may include both hollow and solid portions, as long as the support member 34 provides the desired strength as a platform. The cross-section of the support member 34 may be the same size and shape along its entire length, or the shape and/or size may vary along its length. For one example of a varying cross-section, the middle portion of the support member 34 may be relatively large compared to the size of the ends, such as a large cylinder that tapers down at one or both ends to relatively small cylinders that include a feature for attaching permanently or removably to a surgical frame. In another example, the cross-section of each of the ends of the support member 34 may be different from each other, such as would be the case if the ends are attachable in different ways to a surgical frame (e.g., one end is permanently attached while the other end is removably attached). In any case, it is preferred that the shape of the support member 34 both provide the necessary structural support for the surgery and also be comfortable for the surgeon who contacts it. The shape of the support member 34 also preferably does not interfere with the surgical process.

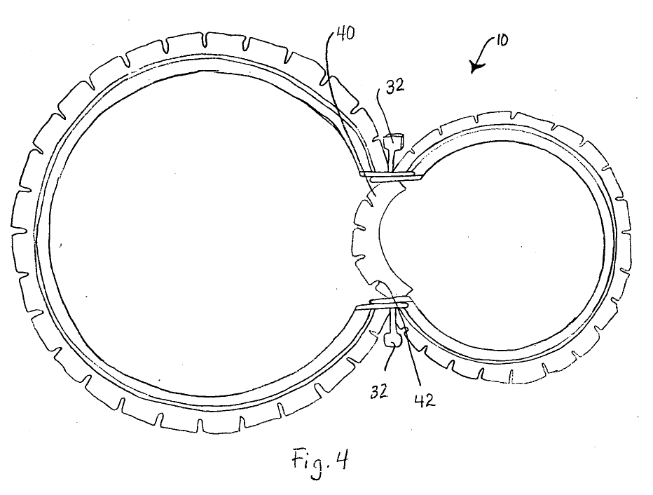

FIG. 4 illustrates the surgical frame 10 of FIG. 1 including another embodiment of a support member of the present invention. In particular, the frame 10 includes a curved support member 40 that is preferably generally shaped to fit the anatomy of the patient. The support member 40 is relatively flat and can optionally be made of the same material from which the frame 10 is made. The support member 40 also optionally includes a plurality of notches 42 along at least one of its edges. These notches 42 provide the surgeon with additional locations to which the elastic stays, sutures, or other surgical components may be attached, if desired. Thus, the support member 40 can provide the dual function of being a supporting platform during the surgical process while also serving as a frame piece to which elastic stays can be attached. This support member 40 may be molded as a portion of the frame 10 or otherwise permanently attached to the frame 10, but may instead be attachable and detachable from the frame.

One example of such a non-permanent attachment of the support member 40 to the frame 10 is with the use of the connection members 32, each of which may be provided with an elongated portion that is long enough to be threaded or pressed into opposite sides of the support member 40 to secure it within the frame 10. In this case, the support member 40 would advantageously be provided with either holes or detents that can accept the end of the connection members 32. Another example of a non-permanent attachment of the support member 40 to the frame 10 is to provide one of the support member 40 and the frame 10 with a detent and the other of the support member and the frame 10 with a corresponding protrusion that can be accepted within the detent. The support member 40 can then be pressed into the frame 10 using similar techniques to those described above relative to the support member installation techniques of FIG. 3 to achieve a type of snap-fit connection. Either of these configurations should therefore allow removal of the support member 40 from the frame before, during, or after the surgery.

Further, the support member 40 may be attached to the frame 10 so that it can pivot with respect to the frame 10. The support member 40 can thus be pivoted so that the surfaces of the support member 40 are in a position that best matches the contours of the surgical site.

Any of the support members or platforms of the present invention may be a solid member that is made generally of the same material as the frame 10 to which it attaches, or may be made of a different material. In either case, the material from which the support member 34 is made is preferably strong enough to provide the desired support for the surgical procedure, while providing a desired amount of rigidity or flexibility, depending on the particular application. That is, the support member 34 should be designed to provide resistance against excessive deflection during the procedure to thereby provide the surgeon with a stable platform while cutting, suturing, installing devices and/or appliances, or performing other surgical procedures. Thus, the material chosen for the support member 34 should be considered for its design in that the size of the member 34 can generally be smaller if the material from which it is made is inherently strong (e.g., metal) and it is desired for the member 34 to be rigid. Conversely, the size of the member 34 will generally be larger if the material used is not as strong (i.e., plastic or rubber) and it is desired for the member 34 to be generally rigid. In cases where some amount of deflection of the support member 34 is acceptable, the member 34 may be made of a material that is semi-rigid. In other cases, it may be acceptable or desirable for the member 34 to be relatively flexible or even malleable so that the surgeon can mold or shape the support member 34 to accommodate a specific patient's anatomy. In addition, a malleable or flexible support member 34 can allow the surgeon to reconfigure the support member 34 to provide better access to the surgical site throughout the surgery.

The support members 34, like the surgical frames to which they are attached, may be reusable and made of a material that can be sterilized at the point of use, such as aluminum, stainless steel, titanium, other medical-grade metals, and the like. Alternatively, the support members 34, like the surgical frames to which they are attached, may instead be disposable and therefore made of a material that is not designed to be sterilizable at the surgical site, such as plastic or resin materials, plastic-coated metals, compound materials, and the like. In cases where the support member 34 is to be flexible or malleable, the support member 34 may be made of a rubber material or the like.

The support member 34 may be attached to the frame 10 at a different orientation than perpendicular to the extension portions 28, 30 (as shown) on both sides of the frame. For example, the support member 34 may be positioned to be entirely within the first frame portion 12 or entirely within the second frame portion 14, and/or may be positioned at an angle relative to the inner area 16 of the frame.

In some cases, it may be desirable to use more than one of the platforms or support members described herein within a single surgical frame. The frame would thus need to be designed for attachment of support members in more than one location, such as additional extending members or other attachment surfaces to which the support members can be secured. This may be particularly useful with a relatively large frame that has a large inner area surrounding a relatively large surgical site to provide the surgeon with an additional support member that can be used as a platform during the surgery. With this configuration, it is contemplated that the surgeon could choose to use more than one support member in the frame throughout the surgery, or that the surgeon only uses one support member at a time and removes any support members that are not in current use during the surgery.

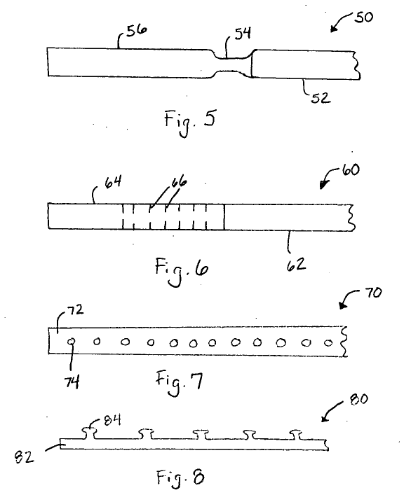

FIG. 5 illustrates one end of an embodiment of a support strap 50 that can be used as a platform within a surgical frame, such as frame 10 described herein. The support strap 50 may be used, for example, to support the penis in a position that makes dissection and exposure of the corpora easier. The strap 50 includes a first portion 52, a second portion 56, and a connecting portion 54 between the first and second portions 52, 56. The three portions 52, 54, 56 may be made of the same or different materials. Each of the three portions may have a different cross-sectional shape and size, or it is possible that the first and second portions 52, 56 have the same cross-sectional shape and size, while the connecting portion 54 has a cross-section that has the same or different shape as the portions 52, 56, but has a smaller size cross-sectional area. For example, the second portion 56 may be oval or elliptical in shape, which tapers to a cylindrical shape for the connecting portion 54. This connecting portion 54 is used as an engagement feature at the end of the main support region (i.e., the first portion 52) to lock the strap 50 within the notches of the surgical frame. The first portion 52 is preferably designed to be strong enough to provide the support necessary for the surgical procedures being performed and/or to support a body organ. Because the connecting portion 54 can engage with any number of notches in a surgical frame, the strap 50 further provides the additional advantage of allowing the surgeon to adjust the tension on the strap 50 by repositioning the connecting portion within different notches in the surgical frame.

The support strap 50 preferably includes the same or a similar configuration (i.e., a connecting portion with a relatively small cross-sectional area) on both ends of the strap 50 so that both ends can engage with the notches of the surgical frame in a wide variety of positions within the frame. That is, the support strap 50 will preferably be positioned to span a portion of the open inner area of a surgical frame. For example, the support strap 50 may be positioned generally in the same area as the support member 34 of FIG. 1, or may instead be positioned further toward one of the ends of the surgical frame. This support strap 50 thus provides the surgeon with a platform or support surface that can be uniquely positioned in whatever areas the surgeon desires additional support. It is further contemplated, however, that only one end of the support strap 50 is free from the surgical frame and has a connecting portion 54, while the other end of the strap 50 is permanently or detachably connected to some part of the surgical frame. In this case, the end of the strap 50 that is not attached to the surgical frame can be positioned as desired relative to the connected end of the strap 50 with its connecting portion 54 positioned within a notch of the surgical frame.

FIG. 6 illustrates one end of a support strap 60 that can be used as a platform within a surgical frame, such as frame 10 described herein. The support strap 60 may be used, for example, to support the penis in a position that makes dissection and exposure of the corpora easier. The strap 60 includes a first portion 62, a second portion 64, and a plurality of gaps or notches 66 spaced along a portion of the length of the second portion 64. The first and second portions 62, 64 may be made of the same or different materials. The notches 66 are preferably spaced at an equal distance from each other, but it is also possible that the notches 66 are irregularly or differently spaced along a portion of the length of the second portion 64. The notches 66 may be provided along both edges of the second portion 64, as shown, with the notches 66 on each edge of the second portion 64 preferably being aligned with a similar notch or gap on the opposite edge of the second portion 64. Alternatively, the notches 66 are not aligned with each other on opposite edges of the strap 60. In any case, the notches 66 are designed so that they will widen or open when the strap 60 is stretched so that they can engage with notches in a surgical frame for attachment of the strap 60 to the frame in a desired location. In addition, it is possible that this type of notch configuration can also be utilized on a portion of elastic retractor stays for attachment of the stays to a surgical frame.

Each of the first and second portions 62, 64 may have the same or a different cross-sectional shape and size. For example, the second portion 64 may be oval or elliptical in shape while the first portion 62 is a generally rectangular flat piece. The notches 66 are designed to be used as an engagement feature at the end of the main support region (i.e., the first portion 62) to lock the strap 60 within the notches of a surgical frame. The first portion 62 is preferably designed to be strong enough to provide the support necessary for the surgical procedures being performed and/or to support a body organ. Because the notches 66 of the strap 60 can engage with any number of notches in a surgical frame, the strap 60 further provides the additional advantage of allowing the surgeon to adjust the tension on the strap 60 by repositioning the connecting portion within different notches in the surgical frame.

The support strap 60 preferably includes the same or a similar configuration (i.e., a portion that includes notches or gaps along one or both edges) on both ends of the strap 60 so that both ends can engage with the notches of the surgical frame in a wide variety of positions within the frame. That is, the support strap 60 will preferably be positioned to span a portion of the open inner area of a surgical frame. For example, the support strap 60 may be positioned generally in the same area as the support member 34 of FIG. 1, or may instead be positioned further toward one of the ends of the surgical frame. This support strap 60 thus provides the surgeon with a platform or support surface that can be uniquely positioned as a support surface in whatever areas the surgeon desires such a support. It is further contemplated, however, that only one end of the support strap 60 is free from the surgical frame, while the other end of the strap 60 is permanently or detachably connected to some part of the surgical frame. In this case, the end of the strap 60 that is not attached to the surgical frame can be positioned as desired relative to the connected end of the strap 60 with a notch or gap 66 positioned within a notch of the surgical frame.

FIG. 7 illustrates one end of another embodiment of a support strap 70 that can be used as a platform within a surgical frame, such as frame 10 described herein. The strap 70 includes a planar portion 72 with a plurality of holes 74 spaced along at least a portion of its length. These holes 74 are sized and shaped to fit over protrusions that extend from a surgical frame for attachment of the strap 70 to the frame. To use this strap 70, the surgeon would pull the strap 70 until it is in its desired location with the desired tension that corresponds with a protrusion from the frame, then press the nearest hole 74 over the protrusion. The holes 74 are preferably spaced at an equal distance from each other, but it is also possible that the holes 74 are irregularly or differently spaced along a portion of the length of the planar portion 72. The protrusions from the frame may include a knob or other end piece that helps to retain the holes in position when placed over the protrusions.

FIG. 8 illustrates one end of a support strap 80 that can be used as a platform within a surgical frame, such as frame 10 described herein. The strap 80 includes a planar portion 82 with a plurality of protrusions 84 spaced along at least a portion of its length. These protrusions 84 are sized and shaped to press into holes within a surgical frame for attachment of the strap 80 to the frame. To use this strap 80, the surgeon would pull the strap 80 until it is in its desired location with the desired tension that corresponds with a hole in the frame, then press the nearest protrusion 84 into the hole. Alternatively, the protrusions 84 may be engaged with existing notches in the surgical frame to secure the strap 80 to the frame. The protrusions 84 are preferably spaced at an equal distance from each other, but it is also possible that the protrusions 84 are irregularly or differently spaced along a portion of the length of the planar portion 82. The protrusions 84 may include a knob or other end piece that helps to retain the protrusions 84 in position when placed in a hole.

When using the frames and support members of the present invention in a surgical process, the surgical frame is positioned so that the sterile-draped incision or surgical area is located within the inner area of the frame for access by the surgeon. Any support members or platforms that are moveable can be positioned in their desired orientation for surgery either before or after positioning the frame around the surgical area. If the platform or support member is designed for supporting a particular body part, that body part should be placed in its desired position before starting the surgery. Elastic stays are then preferably inserted into edges of the tissue on opposite sides of the incision, using equal tension. The elastic stays can then be inserted into the notches of the frame to provide the necessary counter-traction to stabilize the surgical frame for the remainder of surgery so that no additional mechanical support is needed. As dissection progresses, the position of the elastic stays can be advanced to hook into deeper layers of fascia. New stay hooks can be added throughout the process, or those already in place can be repositioned to achieve full, balanced retraction from any desired angle. For closing of the wound after the surgery is complete, it is preferable to maintain constant, opposing tension of the elastic stays for staples or sutures. Any support members may be added, removed, and/or repositioned throughout the surgical procedure.

The frames used with the present invention can have a wide variety of arrangements for the notches around its periphery, where the notches can be identical in size and shape and spaced evenly around the periphery, or may instead include a wide variety of sized, shaped, and spaced notches around the frame periphery. Several examples of alternative notch configurations on a retractor frame are described in the copending U.S. patent application of the present Assignee filed on even date herewith, having U.S. Ser. No.______, entitled “SURGICAL RETRACTOR HAVING SUTURE CONTROL FEATURES”, Attorney Docket No. AMS0056/US, which is incorporated herein by reference in its entirety.

Another alternative manner of connecting any of the support members of the present invention to a surgical frame utilizes the connection members that attach the first and second frame portions 12, 14 to each other (e.g., connection members 32). In particular, the head portion of one or both of the connection members can include a slot or notch. This slot in the connection member is designed to accept an end portion of a support member to secure the support member to the head of the connection member. For example, the connection member can have a relatively narrow slot in its head portion that is designed to accept the notches 66 of the strap 60. It is noted that the head portion of the connection member can be shaped and sized differently from the connection member 32 illustrated herein, and can be particularly designed to pair with an end of a certain support member for a secure connection of the support member. For example, the connection member can have a hole through which the support member can be threaded, the connection member can include a clamp for grasping and securing an end of a support member, the connection member can have an extending peg or protrusion for accepting a strap with holes, such as strap 70 of FIG. 7, or a wide variety of other known attachment methods and devices can be used for this attachment, where the two connection members may have the same or different connection configurations.

The retractor frames shown and described herein are directed generally to surgical techniques and devices that are used for penile-scrotal surgeries however, the retractor frames and features thereof described relative to the present invention can also be used with retractor frames for other surgeries, such as vaginal, urological, colorectal, perineal, hand, foot, plastic reconstructive, vascular, head and neck, and other soft tissue surgeries. In these cases, the outer periphery of the frame is preferably sized and shaped to accommodate the contours of the part of the body with which it will come in contact. In these cases, any support members or strap components can be positioned along the frame periphery in one or more locations that provide the surgical advantages described above, such as providing a platform with sufficient strength and properly positioned for supporting a particular body part.

The present invention has now been described with reference to several embodiments thereof. The entire disclosure of any patent or patent application identified herein is hereby incorporated by reference. The foregoing detailed description and examples have been given for clarity of understanding only. No unnecessary limitations are to be understood therefrom. It will be apparent to those skilled in the art that many changes can be made in the embodiments described without departing from the scope of the invention. Thus, the scope of the present invention should not be limited to the structures described herein, but only by the structures described by the language of the claims and the equivalents of those structures.

1. A surgical retractor system comprising:
an annular frame having an inner edge and an outer edge having a plurality of spaced notches, wherein the inner edge defines an inner area of the frame; and
at least one support member having a first end, a second end, and first and second edges extending between the first and second ends, wherein the first and second ends are attached to the annular frame so that at least a portion of the support member between the first and second ends extends over at least a portion of the inner area of the frame, and wherein at least one of the first and second edges comprises at least one notch.

2. The surgical retractor system of claim 1, wherein at least one notch of the support member is engaged with at least one notch in the outer edge of the annular frame.

3. The surgical retractor system of claim 1, wherein at least one notch of the support member is in the portion of the support member that extends over at least a portion of the inner area of the frame.

4. The surgical retractor system of claim 1, wherein the first end of the support member is permanently attached to the annular frame and the second end of the support member is repositionable relative to the first end of the support member.

5. The surgical retractor system of claim 1, wherein the at least one notch of the support member increases in width in response to stretching the support member.

6. The surgical retractor system of claim 1, wherein the support member comprises notches on both the first and second edges.

7. The surgical retractor system of claim 1, wherein the support member comprises a curved portion.

8. The surgical retractor system of claim 1, wherein the support member is pivotable relative to the annular frame.

9. A surgical retractor system comprising:
an annular frame having an inner edge and an outer edge having a plurality of spaced notches, wherein the inner edge defines an inner area of the frame; and
at least one support member having a first end, a second end, and first and second edges extending between the first and second ends, wherein one of the first and second ends is moveably attached to the annular frame and the other of the first and second ends is permanently attached to the annular frame so that at least a portion of the support member between the first and second ends extends over at least a portion of the inner area of the frame

10. The surgical retractor system of claim 9, wherein the annular frame is made of a first material and the support member is made of a second material that is different from the first material.

11. The surgical retractor system of claim 9, wherein the one of the first and second ends that is moveably attached to the frame is pivotably attached to the frame.

12. The surgical retractor system of claim 9, wherein the one of the first and second ends that is moveably attached to the frame is removably attached to the annular frame.

13. The surgical retractor system of claim 9, wherein the support member is a permanent part of the annular frame.

14. The surgical retractor system of claim 9, wherein the support member is formed as a unitary member with the annular frame.

15. The surgical retractor system of claim 9, wherein the support member is relatively malleable for shaping the support member to a desired surgical configuration.

16. A method of positioning a surgical retractor system for a surgical procedure, comprising the steps of:
providing an annular frame having an inner edge and an outer edge having a plurality of spaced notches, wherein the inner edge defines an inner area of the frame,
attaching a support member having a first end, a second end and first and second edges extending between the first and second ends to the annular frame so that at least a portion of the support member between the first and second ends extends over at least a portion of the inner area of the frame, wherein at least one of the first and second edges comprises a notch; and
positioning the annular frame with attached support member over a surgical area of a patient.

17. The method of claim 16, wherein the step of attaching a support member to the annular frame further comprises engaging at least one notch of the support member with at least one notch of the annular frame.
